(19) United States
(12) Patent Application Publication
(10) Pub. No.: US 2025/0205049 A1
(43) Pub. Date: Jun. 26, 2025

(54) TAVI Motorized Handle Concepts

(71) Applicant: St. Jude Medical, Cardiology Division, Inc., St. Paul, MN (US)

(72) Inventors: Peter J. Ness, Minneapolis, MN (US); Michael Shane Morrissey, St. Paul, MN (US); Robert Schulzetenberg, Columbia Heights, MN (US); Corey Daniel Brown, Andover, MN (US); Parker Blezek, Minneapolis, MN (US); David Copeland, Minnetonka, MN (US)

(73) Assignee: St. Jude Medical, Cardiology Division, Inc., St. Paul, MN (US)

(21) Appl. No.: 18/969,995

(22) Filed: Dec. 5, 2024

Related U.S. Application Data
(60) Provisional application No. 63/615,326, filed on Dec. 28, 2023.
(60) Provisional application No. 63/613,434, filed on Dec. 21, 2023.

(51) Int. Cl. A61F 2/24

(52) U.S. Cl. CPC A61F 2/2433

(57) ABSTRACT

In some embodiments, delivery device includes a delivery catheter, and a handle coupled to the delivery catheter, the handle having a housing and including at least two actuators, each of the at least two actuators being configured to control one of three primary functions, the three primary functions including (1) delivery catheter deflection, (2) delivery catheter axial position fine adjustment, and (3) delivery catheter rotation to achieve native valve commissural alignment.

### CROSS-REFERENCE TO RELATED APPLICATIONS

This application claims priority to U.S. Provisional Patent Application No. 63/613,434, filed Dec. 21, 2023, and U.S. Provisional Patent Application No. 63/615,326, filed Dec. 28, 2023, the disclosures of which are hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves include surgical heart valves, as well as collapsible and expandable heart valves intended for transcatheter aortic valve replacement or implantation (“TAVR” or “TAVI”) or transcatheter mitral valve replacement (“TMVR”). Surgical or mechanical heart valves may be sutured into a native annulus of a patient during an open-heart surgical procedure, for example. Collapsible and expandable heart valves may be delivered into a patient via a delivery apparatus such as a catheter to avoid a more invasive procedure such as full open-chest, open-heart surgery. As used herein, reference to a “collapsible and expandable” heart valve includes heart valves that are formed with a small cross-section that enables them to be delivered into a patient through a catheter in a minimally invasive procedure, and then expanded to an operable state once in place, as well as heart valves that, after construction, are first collapsed to a small cross-section for delivery into a patient and then expanded to an operable size once in place in the valve annulus.

Collapsible and expandable prosthetic heart valves typically take the form of a one-way valve structure (often referred to as a valve assembly) mounted within an expandable frame (the terms “stent” and “frame” may be used interchangeably herein). In general, these collapsible and expandable heart valves include a self-expanding, mechanically-expandable, or balloon-expandable frame, often made of nitinol or another shape-memory metal or metal alloy (for self-expanding frames) or steel or cobalt chromium (for balloon-expandable frames). The one-way valve assembly mounted to/within the stent includes one or more leaflets and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help prevent leakage around the outside of the valve (the latter known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon-expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

Proper controlled deployment of a medical device, such as a prosthetic heart device, is important to ensure positive clinical outcomes. Additionally, controlled and easy deployment would be helpful to an operator, especially during high stress or complex medical procedures.

### SUMMARY OF THE DISCLOSURE

In some embodiments, delivery device includes a delivery catheter, and a handle coupled to the delivery catheter, the handle having a housing and including at least two actuators, each of the at least two actuators being configured to control one of three primary functions, the three primary functions including (1) delivery catheter deflection, (2) delivery catheter axial position fine adjustment, and (3) delivery catheter rotation to achieve native valve commissural alignment.

In some embodiments, a delivery device includes a delivery catheter, and a handle having a housing and including a first actuator configured to control delivery catheter rotation relative to the handle to achieve native valve commissural alignment; and a second actuator configured to control (a) delivery catheter deflection, or (b) delivery catheter axial position fine adjustment.

### BRIEF DESCRIPTION OF THE DRAWINGS

FIG. 1 is a perspective view of an example of a prosthetic heart valve.

FIG. 2 is a front view of an example of a section of the frame of the prosthetic heart valve of FIG. 1, as if cut longitudinally and laid flat on a table.

FIG. 3 is a front view of an example of a prosthetic leaflet of the prosthetic heart valve of FIG. 1, as if laid flat on a table.

FIG. 4 is a top view of the prosthetic heart valve of FIG. 1 mounted on an example of a portion of a delivery system.

FIG. 5 is an enlarged view of the handle of the delivery system shown in FIG. 4.

FIG. 6 is an enlarged view of a distal end of the delivery system shown in FIG. 4.

FIG. 7 is a top view of an example of a balloon catheter when the balloon is inflated.

FIG. 8 is a top view of an example of an inflation system for use with a delivery system similar to that shown in FIG. 4.

FIG. 9 is a side view of the inflation system of FIG. 8.

FIG. 10 is a perspective view of a connection between the inflation system of FIGS. 8-9 and the handle of the delivery system of FIG. 4.

FIG. 11 is a flowchart showing exemplary steps in a procedure to implant the prosthetic heart valve of FIG. 1 into a patient using the delivery system of FIG. 4.

FIG. 12 is a schematic representation of one example of a handle having three actuators.

FIG. 13 is a schematic representation of one example of a handle having a directional D-pad.

FIG. 14 is a schematic representation of one example of a handle having a potentiometer and two isolated buttons.

FIG. 15 is a schematic representation of one example of a handle having a rocker switch and a potentiometer.

FIG. 16 is a schematic representation of one example of a handle having a rocker switch and two adjacent buttons.

FIG. 17 is a schematic representation of one example of a handle having two perpendicular toggle switches.

FIG. 18 is a schematic representation of one example of a handle having four actuators.

FIG. 19 is a schematic representation of one example of a handle having a display window.

FIG. 20 are examples of various information and messages that can be shown in the display window of FIG. 19.

### DETAILED DESCRIPTION OF THE DISCLOSURE

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing valves disclosed herein. However, it should be noted that the use of the valve is not limited to the intended position and orientation but may be deployed in any type of lumen or passageway. For example, although prosthetic heart valves are described herein as prosthetic aortic valves, those same or similar structures and features can be employed in other heart valves, such as the pulmonary valve, the mitral valve, or the tricuspid valve. Further, the term “proximal,” when used in connection with a delivery device or system, refers to a position relatively close to the user of that device or system when it is being used as intended, while the term “distal” refers to a position relatively far from the user of the device. In other words, the leading end of a delivery device or system is positioned distal to the trailing end of the delivery device or system, when the delivery device is being used as intended. As used herein, the terms “substantially,” “generally,” “approximately,” and “about” are intended to mean that slight deviations from absolute are included within the scope of the term so modified. As used herein, the prosthetic heart valves may assume an “expanded state” and a “collapsed state,” which refer to the relative radial size of the stent.

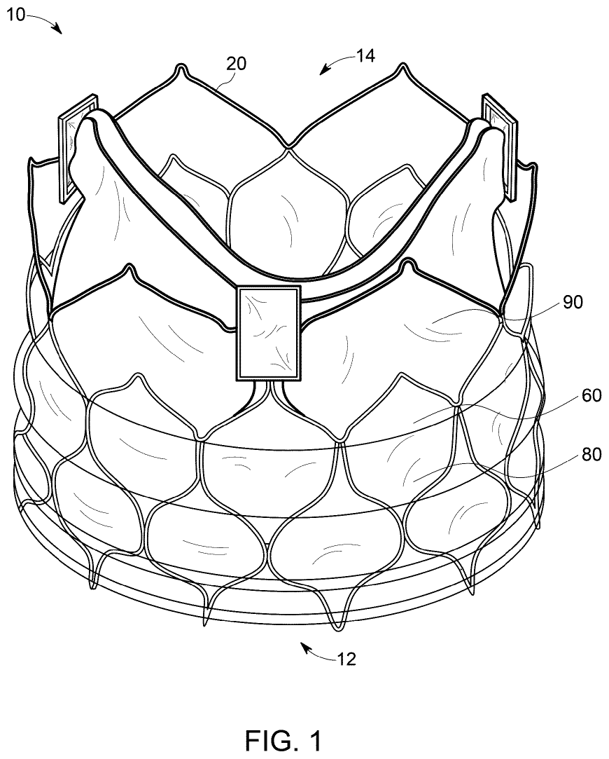

FIG. 1 is a perspective view of one example of a prosthetic heart valve 10. Prosthetic heart valve 10 may be a balloon-expandable prosthetic aortic valve, although in other examples it may be a self-expandable or mechanically-expandable prosthetic heart valve, intended for replacing a native aortic valve or another native heart valve. Prosthetic heart valve 10 is shown in an expanded condition in FIG. 1. Prosthetic heart valve 10 may extend between an inflow end 12 and an outflow end 14. Prosthetic heart valve 10 may include a collapsible and expandable frame 20, an inner cuff or skirt 60, an outer cuff or skirt 80, and a plurality of prosthetic leaflets 90. As should be clear below, prosthetic heart valve 10 is merely one example of a prosthetic heart valve, and other examples of prosthetic heart valves may be suitable for use with the concepts described below.

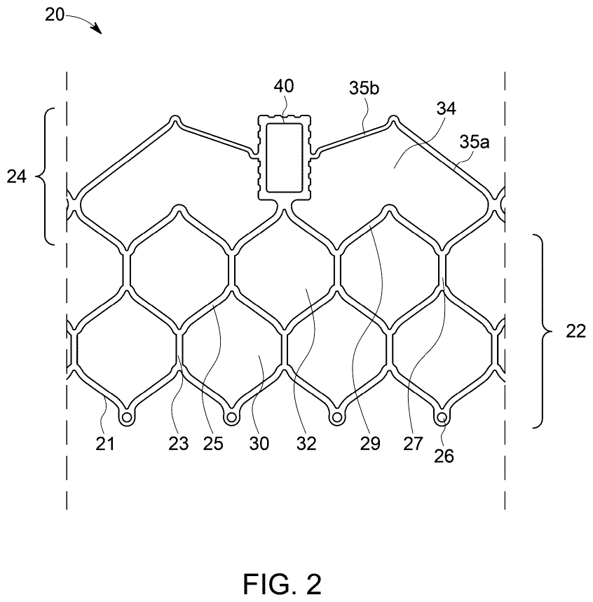

FIG. 2 is a front view of an example of a section of the frame 20 of prosthetic heart valve 10, as if cut longitudinally and laid flat on a table. The section of frame 20 in FIG. 2 may represent approximately one-third of a complete frame, particularly if frame 20 is used in conjunction with a three-leaflet prosthetic heart valve. In the illustrated example, frame 20 is a balloon-expandable stent and may be formed of stainless steel or cobalt-chromium, and which may include additional materials such as nickel and/or molybdenum. However, in some embodiments the stent may be formed of a shape memory material such as nitinol or the like. The frame 20, when provided as a balloon-expandable frame, is configured to collapse upon being crimped to a smaller diameter and/or expand upon being forced open, for example via a balloon within the frame expanding, and the frame will substantially maintain the shape to which it is modified when at rest.

Frame 20 may include an inflow section 22 and an outflow section 24. The inflow section 22 may also be referred to as the annulus section. In one example, the inflow section 22 includes a plurality of rows of generally hexagon-shaped cells. For example, the inflow section 22 may include an inflow-most row of hexagon-shaped cells 30 and an outflow-most row of hexagon-shaped cells 32. The inflow-most row of hexagonal cells 30 may be formed of a first circumferential row of angled or zig-zag struts 21, a second circumferential row of angled or zig-zag struts 25, and a plurality of axial struts 23 that connect the two rows. In other words, each inflow-most hexagonal cell 30 may be formed by two angled struts 21 that form an apex pointing in the inflow direction, two angled struts 25 that form an apex pointing in the outflow direction, and two axial struts that connect the two angled struts 21 to two corresponding angled struts 25. The outflow-most row of hexagonal cells 32 may be formed of the second circumferential row of angled or zig-zag struts 25, a third circumferential row of angled or zig-zag struts 29, and a plurality of axial struts 27 that connect the two rows. In other words, each outflow-most hexagonal cell 32 may be formed by two angled struts 25 that form an apex pointing in the inflow direction, two angled struts 29 that form an apex pointing in the outflow direction, and two axial struts that connect the two angled struts 27 to two corresponding angled struts 29. It should be understood that although the term “outflow-most” is used in connection with hexagonal cells 32, additional frame structure, described in more detail below, is still provided in the outflow direction relative to the outflow-most row of hexagonal cells 32.

In the illustrated embodiment, assuming that frame 20 is for use with a three-leaflet valve and thus the section shown in FIG. 2 represents about one-third of the frame 20, each row of cells 30, 32 includes twelve individual cells. However, it should be understood that more or fewer than twelve cells may be provided per row of cells. Further, the inflow or annulus section 22 may include more or fewer than two rows of cells. Still further, although cells 30, 32 are shown as being hexagonal, the some or all of the cells of the inflow section 22 may have other shapes, such as diamond-shaped, chevron-shaped, or other suitable shapes. In the illustrated embodiment, every cell 30 in the first row is structurally similar or identical to every other cell 30 in the first row, every cell 32 in the second row is structurally similar or identical to every other cell 32 in the second row, and every cell 30 in the first row is structurally similar or identical (excluding the aperture 26) to every cell 32 in the second row. However, in other examples, the cells in each row are not identical to every other cell in the same row or in other rows.

An inflow apex of each hexagonal cell 30 may include an aperture 26 formed therein, which may accept sutures or similar features which may help couple other elements, such as an inner cuff 60, outer cuff 80, and/or prosthetic leaflets 90, to the frame 20. However, in some examples, one or more or all of the apertures 26 may be omitted.

Still referring to FIG. 2, the outflow section 24 of the frame 20 may include larger cells 34 that have generally asymmetric shapes. For example, the lower or inflow part of the larger cells 34 may be defined by the two upper struts 29 of a cell 32, and one upper strut 29 of each of the two adjacent cells 32. In other words, the lower end of each larger cell 34 may be formed by a group of four consecutive upper struts 29 of three circumferentially adjacent cells 32. The tops of the larger cells 34 may each be defined by two linking struts 35a, 35b. The first linking strut 35a may couple to a top or outflow apex of a cell 32 and extend upwards at an angle toward a commissure attachment feature (“CAF”) 40. The second linking strut 35b may extend from an end of the first linking strut 35a back downwardly at an angle and connect directly to the CAF 40. To the extent that the larger cells 34 include sides, a first side is defined by a portion of the CAF 40, and a second side is defined by the connection between first linking strut 35a and the corresponding upper strut 29 of the cell 32 attached to the first linking strut 35a.

The CAF 40 may generally serve as an attachment site for leaflet commissures (e.g. where two prosthetic leaflets 90 join each other) to be coupled to the frame 20. In the illustrated example, the CAF 40 is generally rectangular and has a longer axial length than circumferential width. The CAF 40 may define an interior open rectangular space. The struts that form CAF 40 may be generally smooth on the surface defining the open rectangular space, but some or all of the struts may have one or more suture notches on the opposite surfaces. For example, in the illustrated example, CAF 40 includes two side struts (on the longer side of the rectangle) and one top (or outflow) strut that all include alternating projections and notches on their exterior facing surfaces. These projections and notches may help maintain the position of one or more sutures that wrap around these struts. These sutures may directly couple the prosthetic leaflets 90 to the frame 20, and/or may directly couple an intermediate sheet of material (e.g. fabric or tissue) to the CAF 40, with the prosthetic leaflets 90 being directly coupled to that intermediate sheet of material. In some embodiments, tabs or ends of the prosthetic leaflets 90 may be pulled through the opening of the CAF 40, but in other embodiments the prosthetic leaflets 90 may remain mostly or entirely within the inner diameter of the frame 20. It should be understood that balloon-expandable frames are typically formed of metal or metal alloys that are very stiff, particularly in comparison to self-expanding frames. At least in part because of this stiffness, although the prosthetic leaflets 90 may be sutured or otherwise directly coupled to the frame at the CAFs 40, it may be preferable that most or all of the remaining portions of the prosthetic leaflets 90 are not attached directly to the frame 20, but are rather attached directly to an inner skirt 60, which in turn is directly connected to the frame 20. Further, it should be understood that other shapes and configurations of CAFs 40 may be appropriate. For example, various other suitable configurations of frames and CAFs are described in greater detail in U.S. Provisional Patent Application No. 63/579,378, filed Aug. 29, 2023 and titled “TAVI Deployment Accuracy-Stent Frame Improvements,” the disclosure of which is hereby incorporated by reference herein.

With the example described above, frame 20 includes two rows of hexagon-shaped cells 30, 32, and a single row of larger cells 34. In a three-leaflet embodiment of a prosthetic heart valve that incorporates frame 20, each row of hexagon-shaped cells 30, 32 includes twelve cells, while the row of larger cells includes six larger cells 34. As should be understood, the area defined by each individual cell 30, 32 is significantly smaller than the area defined by each larger cell 34 when the frame 20 is expanded. There is also significantly more structure (e.g. struts) that create each row of individual cells 30, 32 than structure that creates the row of larger cells 34.

One consequence of the above-described configuration is that the inflow section 22 has a higher cell density than the outflow section 24. In other words, the total numbers of cells, as well as the number of cells per row of cells, is greater in the inflow section 22 compared to the outflow section 24. The configuration of frame 20 described above may also result in the inflow section 22 being generally stiffer than the outflow section 24 and/or more radial force being required to expand the inflow section 22 compared to the outflow section 24, despite the fact that the frame 20 may be formed of the same metal or metal alloy throughout. This increased rigidity or stiffness of the inflow section 22 may assist with anchoring the frame 20, for example after balloon expansion, into the native heart valve annulus. The larger cells 34 in the outflow section 24 may assist in providing clearance to the coronary arteries after implantation of the prosthetic heart valve 10. For example, after implantation, one or more coronary ostia may be positioned above the frame 20, for example above the valley where two adjacent larger cells 34 meet (about halfway between a pair of circumferentially adjacent CAFs 40). Otherwise, one or more coronary ostia may be positioned in alignment with part of the large interior area of a larger cell 34 after implantation. Either way, blood flow to the coronary arteries is not obstructed, and a further procedure that utilizes the coronary arteries (e.g. coronary artery stenting) will not be obstructed by material of the frame 20. Still further, the lower rigidity of the frame 20 in the outflow section 24 may cause the outflow section 24 to preferentially foreshorten during expansion, with the inflow section 22 undergoing a relatively smaller amount of axial foreshortening. This may be desirable because, as the prosthetic heart valve 10 expands, the position of the inflow end of the frame 20 may remain substantially constant relative to the native valve annulus, which may make the deployment of the prosthetic heart valve 10 more precise. This may be, for example, because the inflow end of the frame 20 is typically used to gauge proper alignment with the native valve annulus prior to deployment, so axial movement of the inflow end of the frame 20 relative to the native valve annulus during deployment may make precise placement more difficult.

Referring back to FIG. 1, the prosthetic heart valve 10 may include an inner skirt 60 mounted to the interior surface of frame 20. The inner skirt 60 may be formed of tissue, such as pericardium, although other types of tissue may be suitable. In the illustrated example, the inner skirt 60 is formed of a woven synthetic fabric, such as polyethylene terephthalate (“PET”) or polytetrafluoroethylene (“PTFE”), although other fabrics may be suitable, including fabrics other than woven fabrics. In some examples, the inner skirt 60 has straight or zig-zag shaped inflow and outflow ends that generally follow the contours of the cells 30, 32 of the inflow section 22 of frame 20. Preferably, inner skirt 60 is sutured to the frame 20 along the struts that form cells 30, 32. If apertures 26 are included, inner skirt 60 may also be coupled to frame 20 via sutures passing through apertures 26. Preferably, the inner skirt 60 does not cover (or does not cover significant portions of) the larger cells 34. The inner skirt 60 may be coupled to the frame 20 via mechanisms other than sutures, including for example ultrasonic welding or adhesives. Further, the inner skirt 60 may have shapes other than that shown, and need not have a zig-zag inflow or outflow end, and need not cover every cell in the inflow section 22. In fact, in some examples, the inner skirt 60 may be omitted entirely, with the outer skirt 80 (described in greater detail below) being the only skirt used with prosthetic heart valve 10. If the inner skirt 60 is provided, it may assist with scaling the prosthetic heart valve 10 within the heart, as well as serving as a mounting structure for the prosthetic leaflets 90 (described in greater detail below) within the frame 20.

Still referring to FIG. 1, the prosthetic heart valve 10 may include an outer skirt 60 mounted to the exterior surface of frame 20. The outer skirt 80 may be formed of tissue, such as pericardium, although other types of tissue may be suitable. In the illustrated example, the outer skirt 80 is formed of a woven synthetic fabric, such as PET or PTFE, although other fabrics may be suitable, including fabrics other than woven fabrics. In some examples, the outer skirt 80 has straight or zig-zag inflow end. Preferably, outer skirt 80 is sutured to the frame 20 and/or inner skirt 60 along the inflow edge of the outer skirt 80. If apertures 26 are included, outer skirt 80 may also be coupled to frame 20 via sutures passing through apertures 26. The outer skirt 80 may include a plurality of folds or pleats, such a circumferentially extending folds or pleats. The folds or pleats may be formed in the outer skirt 80 via heat setting, for example by placing the outer skirt 80 within a mold that forces the outer skirt 80 to form folds of pleats, and the outer skirt 80 may be treated with heat so that the outer skirt 80 tends to maintain folds or pleats in the absence of applied forces. The outflow edge of outer skirt 80 may be coupled to the frame 20 at selected, spaced apart locations around the circumference of the frame 20. In some embodiments, the outflow edge of outer skirt 80 may be connected to the inner skirt 60 along a substantially continuous suture line. Some or all of the outer skirt 80 between its inflow and outflow edges may remain not directly couples to the frame 20 or inner skirt 60. Preferably, the outer skirt 80 does not cover (or does not cover significant portions of) the larger cells 34. In use, the outer skirt 80 may directly contact the interior surface of the native heart valve annulus to assist with scaling, including scaling against PV leak. If folds or pleats are included with the outer skirt 80, the additional material of the folds or pleats may help further mitigate PV leak. However, it should be understood that the folds or pleats may be omitted from outer skirt 80, and the outer skirt 80 may have shapes other than that shown. In fact, in some examples, the outer skirt 80 may be omitted entirely, with the inner skirt 60 being the only skirt used with prosthetic heart valve 10. If the inner skirt 60 is omitted, the prosthetic leaflets 90 may be attached directly to the frame 20 and/or directly to the outer skirt 80.

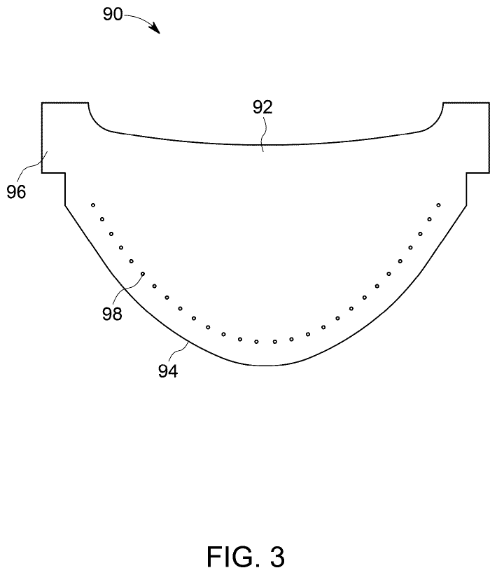

FIG. 3 is a front view of a prosthetic leaflet 90, as if laid flat on a table. In the illustrated example of prosthetic heart valve 10, a total of three prosthetic leaflets 90 are provided, although it should be understood that more or fewer than three prosthetic leaflets may be provided in other example of prosthetic heart valves. The prosthetic leaflet 90 may be formed of a synthetic material, such a polymer sheet or woven fabric, or a biological material, such a bovine or porcine pericardial tissue. However, other materials may be suitable. In on example, the prosthetic leaflet 90 is formed to have a concave free edge 92 configured to coapt with the free edges of the other leaflets to help provide the one-way valve functionality. The prosthetic leaflet 90 may include an attached edge 94 which is attached (e.g. via suturing) to other structures of the prosthetic heart valve 10. For example, the attached edge 94 may be coupled directly to the inner skirt 60, directly to the frame 20, and/or directly to the outer skirt 80. It may be preferable that the attached edge 94 is coupled directly only to the inner skirt 60, which may help reduce stresses on the prosthetic leaflet 90 compared to if the attached edge 94 were coupled directly to the frame 20. In some embodiments, a plurality of holes 98 may be formed along the attached edge 94 (or a spaced distance therefrom), for example via lasers. If included, the holes 98 may be used to receive sutures therethrough, which may make it easier to couple the prosthetic leaflet 90 to the inner skirt 60 during manufacturing. For example, the holes 98 may serve as guides if suturing is performed manually, and if the positions of the holes 98 are controlled via the use of layers, the holes 98 may be consistently placed among different prosthetic leaflets 90 to reduce variability between different prosthetic leaflets 90. Laflet tabs 96 may be provided at the junctions between the free edge 92 and the attached edge 94. Each leaflet tab 96 may be joined to a leaflet tab of an adjacent prosthetic leaflet to form prosthetic leaflet commissures, which may be coupled to the frame 20 via CAFs 40.

The prosthetic heart valve 10 may be delivered via any suitable transvascular route, for example transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing or supporting the valve is inserted through the femoral artery and advanced against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstances, under an overlying sheath). Upon arrival at or adjacent to the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

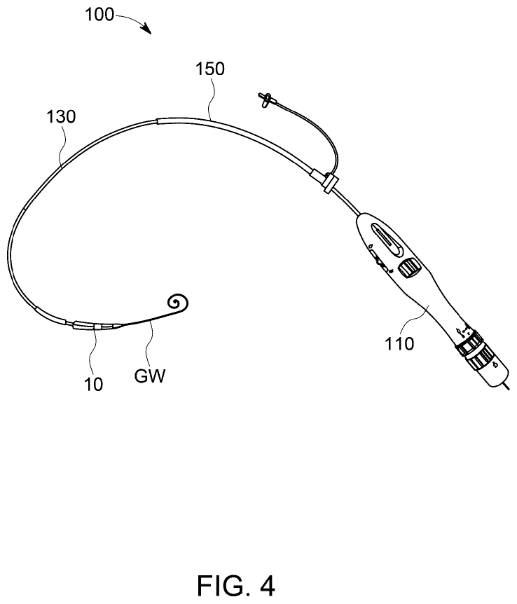

FIG. 4 illustrates one example of a delivery system 100, with the prosthetic heart valve 10 crimped over a balloon on a distal end of the delivery system 100. Although delivery system 100 and various components thereof are described below, it should be understood that delivery system 100 is merely one example of a balloon catheter that may be appropriate for use in delivering and deploying prosthetic heart valve 10.

In some examples, delivery system 100 includes a handle 110 and a delivery catheter 130 extending distally from the handle 110. An introducer 150 may be provided with the delivery system 100. Introducer 150 may be an integrated or captive introducer, although in other embodiments introducer 150 may be a non-integrated or non-captive introducer. In some examples, the introducer 150 may be an expandable introducer, including for example an introducer that expands locally as a large diameter components passes through the introducer, with the introducer returning to a smaller diameter once the large diameter components passes through the introducer. In other examples, the introducer 150 is a non-expandable introducer.

A guidewire GW may be provided that extends through the interior of all components of the delivery system 100, from the proximal end of the handle 110 through the atraumatic distal tip 138 of the delivery catheter 130. The guidewire GW may be introduced into the patient to the desired location, and the delivery system 100 may be introduced over the guidewire GW to help guide the delivery catheter 130 through the patient's vasculature over the guidewire GW.

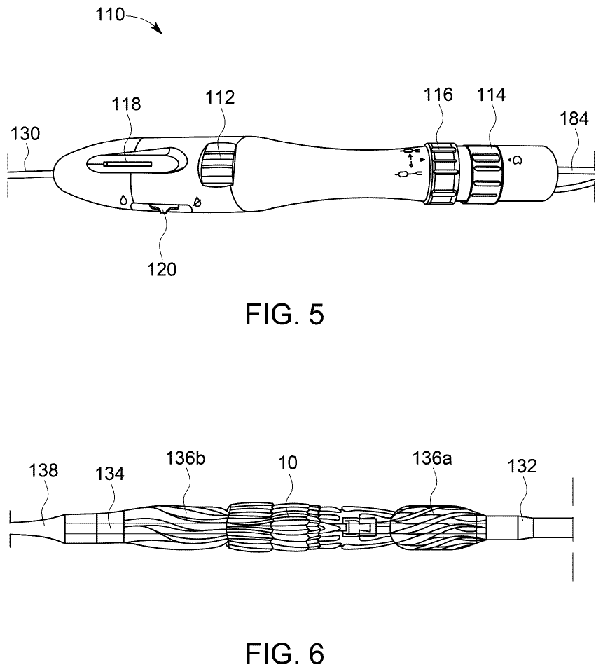

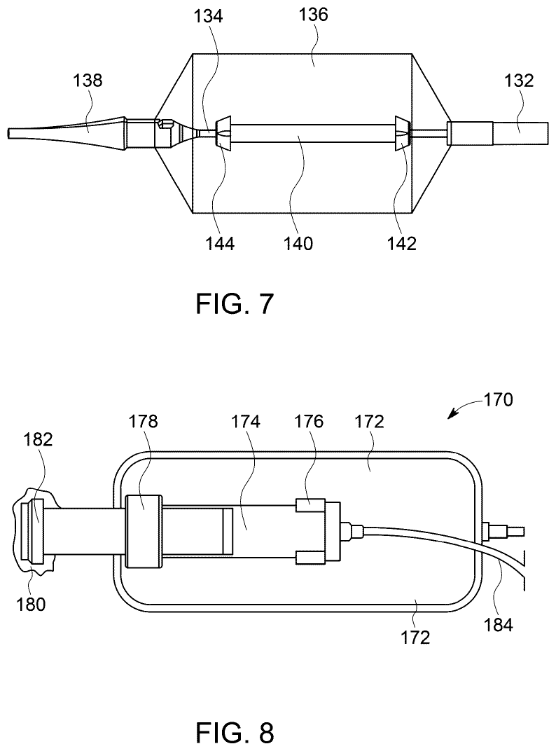

In some examples, the delivery catheter 130 is steerable. For example, one or more steering wires may extend through a wall of the delivery catheter 130, with one end of the steering wire coupled to a steering ring coupled to the delivery catheter 130, and another end of the steering wire operable coupled to a steering actuator on the handle 110. In such examples, as the steering actuator is actuated, the steering wire is tensioned or relaxed to cause deflection or straightening of the delivery catheter 130 to assist with steering the delivery catheter 130 to the desired position within the patient. For example, FIG. 5 is an enlarged view of the handle 110. Handle 110 may include a steering knob 112 that, upon rotation, tensions or relaxes the steering wires to deflect the distal end of the delivery catheter 130. A deflection indicator 118 may be included that shows the extent of the deflection of the distal end of delivery catheter 130. However, it should be understood that the steering functionality may be omitted in some examples, and in other examples steering actuators other than knobs may be utilized. Further, in some examples, including those shown in FIGS. 6-7, the delivery catheter 130 includes an outer catheter 132, and an inner catheter 134. The steering functionality may be provided in either the outer catheter 132, or the inner catheter 134, or in both catheters.

Still referring to FIGS. 4-5, the delivery system 100 may include additional functionality to assist with positioning the prosthetic heart valve 10. For example, in the illustrated example, handle 110 includes a commissure alignment actuator 114, which may be positioned near a proximal end of the handle or at any other desired location. In the illustrated example, the commissure alignment actuator 114 is in the form of a rotatable knob, although other forms may be suitable. The commissure alignment knob 114 may be rotationally coupled to a portion of the delivery catheter 130 supporting the prosthetic heart valve 10. For example, the commissure alignment actuator 114 may be rotationally coupled to an inner catheter 134 which supports the prosthetic heart valve 10 in the crimped condition. With this configuration, rotating the commissure alignment knob 114 may cause the inner catheter 134 to rotate about its longitudinal axis, and thus cause the prosthetic heart valve 10 to rotate about its longitudinal axis. If a commissure alignment actuator 114 is included, it may be used to help ensure that, upon deployment of the prosthetic heart valve 10 into the native valve annulus, the commissures of the prosthetic heart valve are in rotational alignment with respective ones of the native valve commissures (e.g. within +/−2.5 degrees of rotational alignment, within +/−5 degrees of rotational alignment, within +/−10 degrees of rotational alignment, within +/−15 degrees of rotational alignment, etc.). Although commissure alignment actuator 114 is shown in this example as a knob positioned at or near a proximal end of the handle 110, it should be understood that the actuator 114 may take forms other than a knob, may be positioned at other suitable locations, and may be omitted entirely if desired.

Still referring to FIGS. 4-5, the delivery system 100 may include even further functionality to assist with positioning the prosthetic heart valve 10. For example, in the illustrated example, handle 110 includes an axial alignment actuator 116, which may be positioned near a proximal end of the handle, including distal to the commissure alignment actuator 114, or at any other desired location. In the illustrated example, the axial alignment actuator 116 is in the form of a rotatable knob, although other forms may be suitable. The axial alignment knob 116 may be operably coupled to a portion of the delivery catheter 130 supporting the prosthetic heart valve 10. For example, the axial alignment actuator 116 may include internal threads that engage external threads of a carriage that is coupled to an inner catheter 134 which supports the prosthetic heart valve 10 in the crimped condition. In such an example, the carriage may be rotatably fixed to the handle 110. With this configuration, rotating the axial alignment knob 116 may cause the carriage to advance distally or retract proximally as the inner threads of the axial alignment knob 116 mesh with the external threads of the carriage, but the carriage is prevented from rotating. As the carriage advances distally or retracts proximally, the inner catheter 134 may correspondingly advance distally or retract proximally, and thus cause the prosthetic heart valve 10 to advanced distally or retract proximally. It should be understood that, if axial alignment actuator 116 is included, it have a small total range of motion. In other words, the rough or coarse axial alignment between the prosthetic heart valve 10 and native valve annulus may be achieved by physically advancing the entire delivery catheter 130 by pushing it through the vasculature while holding the handle 110. However, for fine and more controlled adjustment of the axial position of the prosthetic heart valve 10 relative to the native valve annulus, which may be performed just prior to or during deployment of the prosthetic heart valve 10, the axial alignment knob 116 may be used. If an axial alignment actuator 116 is included, it may be used to help ensure that, upon deployment of the prosthetic heart valve 10 into the native valve annulus, the inflow end of the of the prosthetic heart valve is in axial alignment with the inflow aspect of the native valve annulus (e.g. within +/−0.5 mm of axial alignment, within +/−1.0 mm of axial alignment, within +/−1.5 mm of axial alignment, within +/−2.0 mm of axial alignment, etc.). Although axial alignment actuator 116 is shown in this example as a knob positioned at or near a proximal end of the handle 110, it should be understood that the actuator 116 may take forms other than a knob, may be positioned at other suitable locations, and may be omitted entirely if desired.

In addition to steering and positioning actuators, delivery system 100 may include a balloon actuator 120. In the illustrated example, balloon actuator 120 is positioned on the handle 110 near a distal end thereof, and is provided in the form of a switch. Balloon actuator 120 may be actuated to cause inflation or deflation of a balloon 136 that is part of the delivery system 100. For example, referring briefly to FIGS. 6-7, the delivery system 100 may include a balloon 136 that overlies a distal end of inner catheter 134 and which receives the prosthetic heart valve 10 in a crimped condition thereon. In the example illustrated in FIG. 6, the balloon 136 includes a proximal pillowed portion 136a, a distal pillowed portion 136b, and a central portion over which the prosthetic heart valve 10 is crimped. The proximal pillow 136a and the distal pillow 136b may form shoulders on each side of the prosthetic heart valve 10, which may help ensure the prosthetic heart valve 10 does not move axially relative to the balloon 136 and/or inner catheter 134 during delivery. The shoulder formed by the distal pillow 136 may also help protect the inflow edge of the prosthetic heart valve 10 from contact with the anatomy during delivery. For example, during a transfemoral delivery, as the distal end of the delivery catheter 130 traverse the sharp bends of the aortic arch (or during initial introduction into the patient), there is a relatively high likelihood the inflow end of the prosthetic heart valve 10 (which is the leading edge during transfemoral delivery) will contact a vessel wall (or a components of an introduction system) causing dislodgment of the prosthetic heart valve 10 relative to the balloon 136. The distal pillow 136 may tend to have an equal or larger outer diameter than the inflow end of the prosthetic heart valve 10 (when the prosthetic heart valve 10 is crimped and the balloon 136 is deflated), which may help ensure the inflow edge of the prosthetic heart valve 10 does not inadvertently contact another structure during delivery. In some examples, the pillowed portions 136a, 136b may be formed via heat setting. Additional related features for use in similar balloon catheter delivery systems are described in greater detail in U.S. Provisional Patent Application No. 63/382,812, filed Nov. 8, 2022 and titled “Prosthetic Heart Valve Delivery and Trackability,” the disclosure of which is hereby incorporated by reference herein.

In order to deploy the prosthetic heart valve 10, the balloon 136 is inflated, for example by actuating the balloon actuator 120 to force fluid (such as saline, although other fluids, including liquids or gases, could be used) into the balloon 136 to cause it to expand, causing the prosthetic heart valve 10 to expand in the process. For example, the balloon actuator 120 may be pressed forward or distally to cause fluid to travel through an inflation lumen within delivery catheter 130 to inflate the balloon 136. FIG. 7 illustrates an example of the balloon 136 after being inflated, with the prosthetic heart valve 10 omitted from the figure for clarity. In the illustrated example, the balloon 136 may be formed to have a distal end that is fixed to a portion of an atraumatic distal tip 138. The distal tip 138 may be tapered to help the delivery catheter 130 move through the patient's vasculature more smoothly. A proximal end of the balloon 136 may be fixed to a distal end of outer catheter 132. The inflation lumen may be the space between the outer catheter 132 and the inner catheter 134, or in other embodiments may be provided in a wall of the inner catheter 134, or in any other location that fluidly connects the interior of the balloon 136 to a fluid source outside of the patient that is operable coupled to the delivery system 100.

Referring to FIG. 7, in some examples, a mounting shaft 140 may be provided on the inner catheter 134. A proximal stop 142 and/or a distal stop 144 may be provided, for example at opposite ends of the mounting shaft 140. If the mounting shaft 140 is included, it may provide a location on which the prosthetic heart valve 10 may be crimped. If the proximal stop 142 and/or distal stop 144 is provided, they may provide physical barriers to the prosthetic heart valve 10 moving axially relative to the balloon 136. In one example, the proximal stop 142 may taper from a larger distal diameter to a smaller proximal diameter, and the distal stop may taper from a larger proximal diameter to a smaller distal diameter. The spacing between the proximal stop 142 and the distal stop 144, if both are included, may be slightly larger than the length of the prosthetic heart valve 10 when it is crimped over mounting shaft 140. However, it should be understood that one or both of the stops 142, 144 may be omitted, and the mounting shaft 140 may also be omitted. If the mounting shaft 140 is included, it is preferably axially and rotationally fixed to the inner catheter 134 so that movement of the inner catheter 134 causes corresponding movement of the mounting member 140, and thus the prosthetic heart valve 10 when mounted thereon.

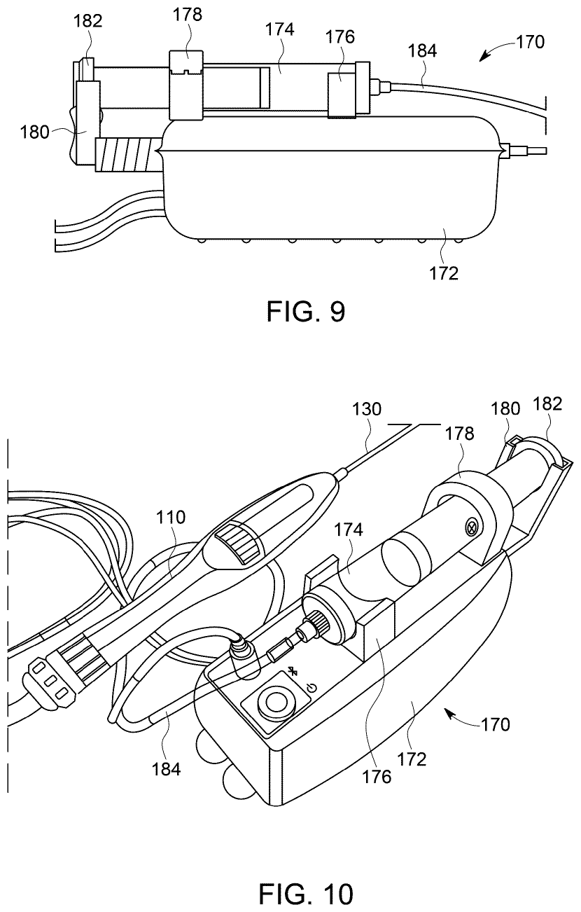

Before describing the use of balloon actuator 120 in more detail, it should be understood that in some embodiments, the balloon actuator 120 may be omitted and instead a manual device, such as a manual syringe, may be provided along with delivery system 100 in order to manually push fluid into balloon 136 during deployment of the prosthetic heart valve 10. However, in the illustrated example of delivery system 100, the balloon actuator 120 provides for a motorized and/or automated (or semi-automated) balloon inflation functionality. For example, FIG. 8 and FIG. 9 illustrate an example of a balloon inflation system 170. Balloon inflation system 170 may include a housing 172 that houses one or more components, which may include a motor, one or more batteries, electronics for control and/or communication with other components, etc. Housing 172 may include one or more fixed cradles to receive a syringe 174. In the illustrated embodiment, a distal cradle 176 is provide with an open “C”- or “U”-shaped configuration so that the distal end of the syringe 174 may be snapped into or out of the distal cradle 176. A proximal cradle 178 may also be provided, which may have a “C”- or “U”-shaped bottom portion hingedly connected to a “C”- or “U”-shaped top portion. This configuration may allow for the proximal end of the outer body of the syringe 174 to be snapped into the bottom portion of proximal cradle 178, and the top portion of proximal cradle 178 may be closed and connected to the bottom portion to fully circumscribe the outer body of the syringe 174 to lock the syringe 174 to the housing 172. It should be understood that more or fewer cradles, of similar or different designs, may be included with housing 172 to help secure the syringe 174 to the housing 172 in any suitable fashion.

The balloon inflation system 170 may include a moving member 180. In the illustrated embodiment, moving member 180 includes a “C”- or “U”-shaped cradle to receive a plunger handle 182 of the syringe 174 therein, the cradle being attached to a carriage that extends at least partially into the housing 172. The carriage of the moving member 180 may be generally cylindrical, and may include internal threading that mates with external threading of a screw mechanism (not shown) within the housing 172 that is operably coupled to a motor. In some embodiments, the carriage may have the general shape of a “U”-beam with the flat face oriented toward the top. The moving member 180 may be rotationally fixed to the housing 172 via any desirable mechanism, so that upon rotation of the screw mechanism by the motor, the moving member 180 advances farther into the housing 172, or retracts farther away from the housing 172, depending on the direction of rotation of the screw mechanism. While the plunger handle 182 is coupled to the moving member 180, advancement of the moving member 180 forces fluid from the syringe 174 toward the balloon 136, while retraction of the moving member 180 withdraws fluid from the balloon 136 toward the syringe 174. It should be understood that the motor, or other driving mechanism, may be located in or outside the housing 172, and any other suitable mechanism may be used to operably couple the motor or other driving mechanism to the moving member 180 to allow for axial driving of the plunger handle 182.

As shown in each of FIG. 8, FIG. 9, and FIG. 10, the distal end of syringe 174 may be coupled to tubing 184 that is in fluid communication with an inflation lumen of delivery catheter 130 that leads to the balloon 136 at or near the distal end of the delivery system 100. Tubing 184 may allow for the passage of the fluid (e.g., saline) from the syringe 174 toward the balloon 136, or for withdrawal of fluid from the balloon 136 toward the syringe 174, for example based on whether the balloon actuator 120 is pressed forward or backward.

Although not separately numbered in FIG. 8, FIG. 9, and FIG. 10, the housing 172 may include one or more cables extending from the housing, for example to allow for transmission of power (e.g. from AC mains or another component with which the cable is coupled) and/or transmission of data, information, control commands, etc. For example, one cable may couple the housing 172 to handle 110 so that controls on the handle 110 (e.g. balloon actuator 120) may be used to activate the balloon inflation system 170 in the desired fashion. Another cable may couple to a computer display or similar device to provide information regarding the inflation of the balloon 136. However, it should be understood that any transmission of data or information may be provided wirelessly instead of via a wired connection, for example via a Bluetooth or other suitable connection. Additional and related features of balloon inflation system 170, related systems, and the uses thereof are described in U.S. patent application Ser. No. 18/311,458, the disclosure of which is hereby incorporated by reference herein.

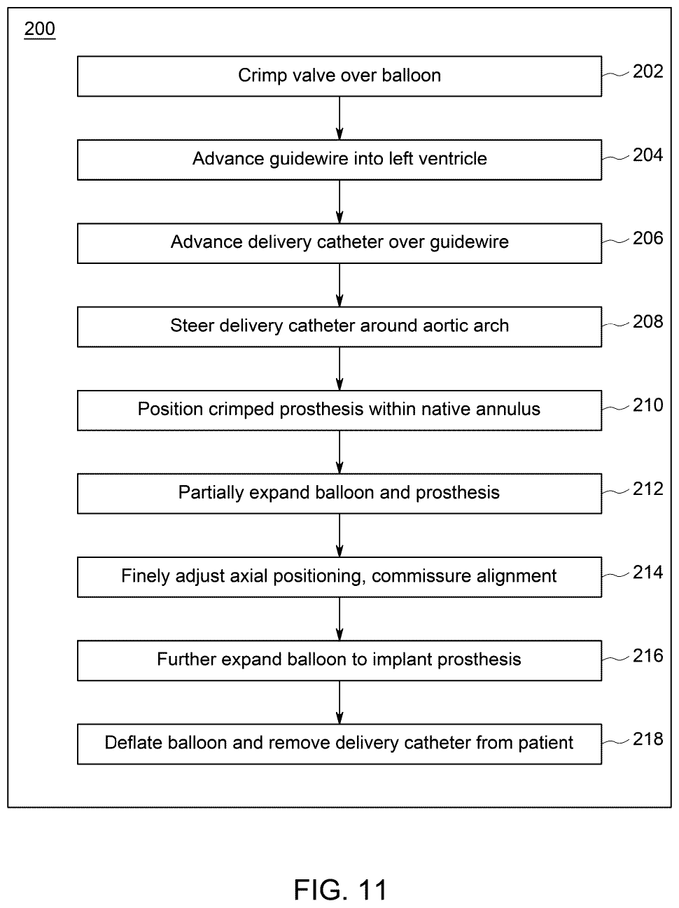

FIG. 11 is a flowchart showing exemplary steps in an implantation procedure 200 to implant the prosthetic heart valve 10 of FIG. 1 into a patient using the delivery system 100 of FIG. 4. However, it should be understood that not all of the steps shown in connection with implantation procedure 200 need to be performed, and various steps not explicitly shown and described in connection with procedure 200 may be performed as part of the implantation procedure. At the beginning of the procedure 200 in step 202, the prosthetic heart valve 10 may be collapsed over or crimped onto balloon 136, with the balloon 136 being mostly or entirely deflated after the crimping procedure. It should be understood that crimping step 202 may be performed at any time prior to the procedure, including at the beginning of the procedure, or at an earlier stage before the delivery system 100 is provided to the end user. In other words, the crimping step 202 may be performed during a manufacturing stage of the delivery system 100 and/or prosthetic heart valve 10. During an early stage of the implantation procedure 200, a guidewire GW may be advanced into the patient in step 204, for example via the femoral artery, around the aortic arch, through the native aortic valve, and into the left ventricle. The guidewire GW may be used as a rail for other devices that need to access this pathway. For example, in step 206, the atraumatic distal tip 138 may be advanced over the proximal end of the guidewire GW, and the delivery catheter 130 may be advanced over guidewire GW toward the native aortic valve. During this initial advancement of the delivery catheter 130 into the patient, the introducer 150 (if included) may be positioned distally, for example so that it covers the prosthetic heart valve 10 or so that it is positioned just proximal to the prosthetic heart valve 10. Advancement of the delivery catheter 130 and introducer 150 may continue until a proximal hub of the introducer is in contact with the patient's skin (or in contact with another device that enters the patient's femoral artery. At this point, the introducer 150 may stop moving axially relative to the patient, with the delivery catheter 130 continuing to advance relative to the introducer 150. If steering capability is provided, the delivery catheter 130 may be steered or deflected at any point to assist with achieving the desired pathway of the delivery catheter 130. As on example, in step 208, the steering knob 112 may be actuated to deflect the distal end of the delivery catheter 130 as it traverses the sharp bends of the aortic arch. Advancement of the delivery catheter 130 may continue in step 210 until the prosthetic heart valve 10, while still crimped or collapsed, is positioned within the native aortic valve annulus. With the desired position achieved, the balloon 136 may be partially inflated, for example by pressing balloon actuator 120 forward, to partially expand the prosthetic heart valve 10 in step 212. In some examples, it is desirable to expand the prosthetic heart valve 10 only partially in step 212, because the position of the prosthetic heart valve 10 (including rotational and/or axial positioning) relative to the native aortic valve annulus may shift during this partial expansion. After the partial expansion of step 212, the user may examine the positioning of the prosthetic heart valve 10 relative to the native aortic valve annulus. If desired, in step 214, the axial positioning of the partially-expanded prosthetic heart valve 10 relative to the native aortic valve annulus may be finely adjusted (e.g. by actuating axial alignment actuator 116) and/or the rotational orientation of the prosthetic heart valve 10 relative to the native aortic valve may be finely adjust (e.g. by actuating commissure alignment actuator 114). When the desired axial alignment is achieve and the desired rotational alignment (e.g. rotational alignment between the prosthetic commissure and the native commissures) is achieved, the balloon 136 may be fully expanded in step 216 to fully expand the prosthetic heart valve 10 and to anchor the prosthetic heart valve 10 in the native aortic valve annulus in the desired position and orientation. After deployment is complete, the balloon 136 may be deflated in step 218, for example by pressing actuating balloon 120 backward, and the delivery catheter 130 and guidewire GW may be removed from the patient to complete the procedure. It should be understood that the nine steps shown in FIG. 11 as part of procedure 200 are merely exemplary of a single example of an implantation procedure, and steps shown may be omitted, steps not shown may be included, and steps may be provided in any order deemed appropriate by the physician and/or medical personnel.

Although various components of a prosthetic heart valve 10 and delivery system 100 are described above, it should be understood that these components are merely intended to provide better context to the systems, features, and/or methods described below. Thus, various components of the systems described above may be modified or omitted as appropriate without affecting the systems, features, and/or methods described below. For example, prosthetic heart valves other than the specific configuration shown and described in connection with FIGS. 1-3 may be used with delivery systems other than the specific configuration shown and described in connection with FIGS. 4-10 as part of an implantation procedure that uses steps other than the specific configuration shown and described in connection with FIG. 11, without affecting the inventive systems, features, and/or methods described below.

As previously noted, controlled deployment of a medical device, such as a prosthetic heart valve is desirable. In some examples, cardiovascular implants may use mechanical means to deploy implants such as stents, transcatheter valves, or repair devices. In some examples for implanting a prosthetic heart valve, a delivery device may provide any of three primary functions and these may include, but are not limited to: (1) catheter deflection used for navigating tortuous anatomy and/or achieving coaxial alignment with a target vessel, and this may be accomplished via a steering wire that is tensioned or relaxed to cause deflection or straightening of the delivery catheter 130 to assist with steering the delivery catheter 130 to the desired position within the patient, (2) catheter axial position fine adjustment to achieve accurate axial implant placement, and/or (3) fine or subtle catheter rotation to achieve native valve commissural alignment. Additional functions may be added to these three primary functions. In some examples, one or more of the three primary functions, along with other features, may be actuated or activated at the delivery system control handle via direct translation, displacement, rotation, a leadscrew mechanism, or cam-driven actuation.

In the instant disclosure, various methods are provided for motorizing and/or digitizing features of medical device (e.g., prosthetic heart valve) delivery or catheter-based devices in general, including the three primary functions described above. In some examples, these devices and methods are intended to improve streamlined ergonomics, reduced procedure time, provide more controlled action, and/or add the ability to incorporate closed loop feedback.

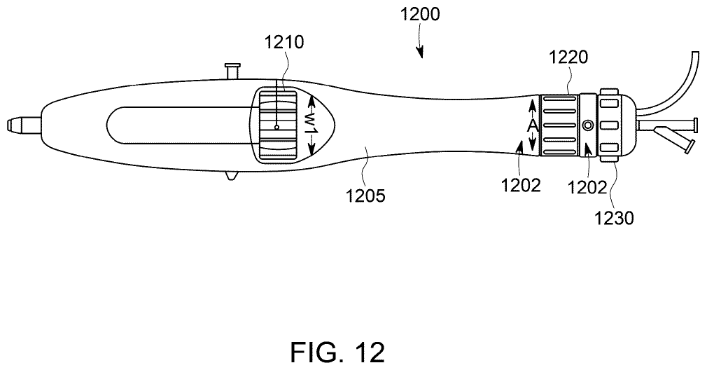

FIG. 12 illustrates one example of a delivery device handle 1200 having a housing 1205 and several actuators for operating the delivery device. In this example, handle 1200 includes a first actuator 1210 in the form of a first wheel disposed adjacent the distal end of the handle, a second actuator 1220 in the form of a second wheel disposed closer to the proximal end of the handle, and a third actuator 1230 in the form of a third wheel disposed proximal to the second actuator. In some examples, first actuator 1210 may be a wheel, rotatable in either direction (along directions wl as shown), to control deflection of a catheter, second actuator 1220 may have a wheel rotatable in the same directions as the first actuator to control fine axial adjustment of the catheter (i.e., axially forward and/or backward), and third actuator 1230 may be rotated along the same directions to control commissural alignment of a prosthetic heart valve or feature of an implant. It will be understood that variations are possible and that the function of the three actuators may be substituted for any of the three primary functions or others (e.g., first actuator 1210 may control fine axial adjustment or commissural alignment, second actuator 1220 may control deflection or commissural alignment, and third actuator 1230 may control deflection or fine axial adjustment, etc.). Markings 1202 (e.g., icons, indicators, pictures and/or text) may be used to signal to the user the primary function associated with each actuator.

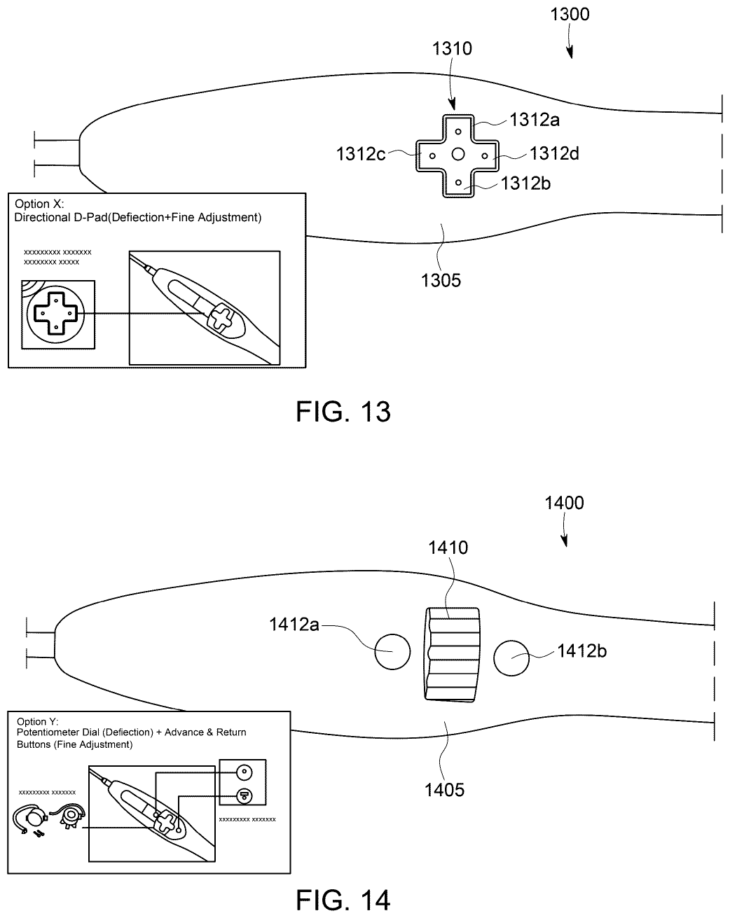

FIG. 13 illustrates an example of a delivery device handle 1300 having a housing 1305 and directional pad 1310 for operating the delivery device. Directional pad 1310 may be a flat, typically thumb-operated, directional control. In this example, and as shown in this orientation, directional pad (or “D-pad”) 1310 includes a first (“up”) internal button 1312a, an opposing second (“down”) internal button 1312b, a third (“left”) internal button 1312c and an opposing fourth (“right”) internal button 1312d, and each internal button may provide a discrete operation. It will be understood that the labels “up,” “down,” “left,” and “right” used above are only used to illustrate the positions of the internal button on the device in the orientation shown, and are not meant to limit the functionality of any button. Directional pad 1310 may be used to actuate or activate different features that may be used simultaneously or in quick sequential order, such as catheter deflection and fine adjustment. For example, one set of internal buttons (e.g., the first and the second internal buttons 1312a, 1312b may be used to control deflection) while another set of internal buttons (e.g., the third and fourth internal buttons 1312c, 1312d) may be used to control fine axial adjustment or vice versa. Additionally, any of three primary functions (1), (2), or (3) above may be mapped to any of the four buttons.

FIG. 14 illustrates an example of a delivery device handle 1400 having a housing 1405 and a first actuator 1410 in the form of a wheel, and two additional actuators 1412a, 1412b in the form of round push buttons for operating the delivery device. In this example, first actuator 1410 may act as a potentiometer to control a first primary function (e.g., catheter deflection), while actuators 1412a, 1412b may be useful as push buttons to control fine axial adjustment (e.g., actuator 1412a used to control fine forward translation of a catheter, while actuator 1412b is used to control fine backward translation of a catheter). First actuator 1410 and/or additional actuators 1412a, 1412b may also, or instead, be used to control commissural alignment. Alternatively, the function of the actuators may be swapped the orientation of the wheel may also, in addition or instead, be changed so that a wheel rotates forward and/or backward to make fine axial translation more intuitive in some examples.

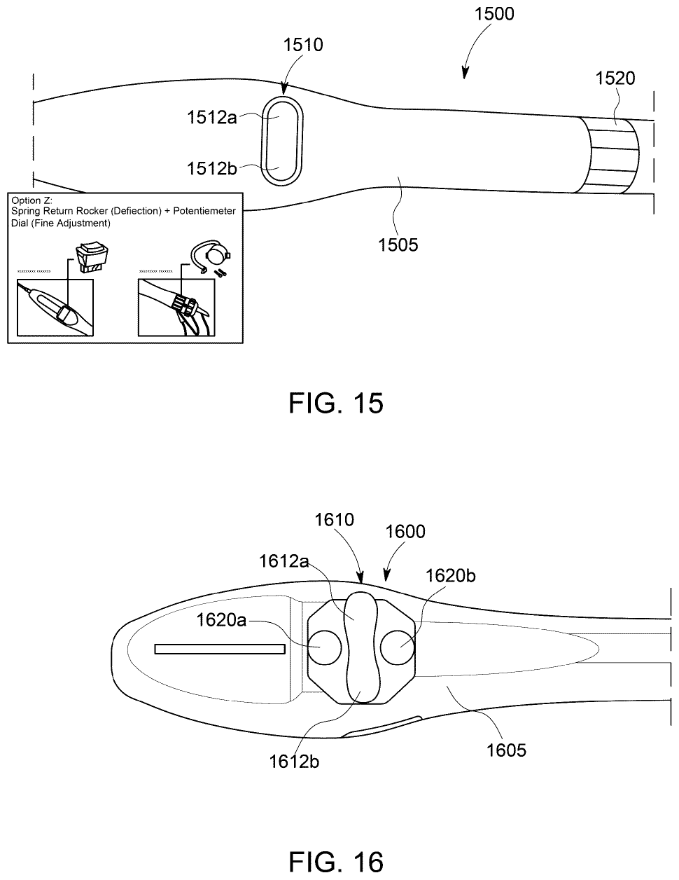

FIG. 15 illustrates an example of a delivery device handle 1500 having a housing 1505 and a first actuator 1510 in the form of a spring-return rocker switch having a first internal button 1512a and a second internal button 1512b, and an additional actuator 1520 in the form of a wheel for operating the delivery device. In this example, first actuator may include a spring-return rocker so that pressing first or second internal buttons 1512a, 1512b toggles or activates one of the primary functions and that the switch returns to a neutral condition where the two internal buttons are level when no force is applied thereto. In this example, first actuator 1510 may control a first primary function (e.g., catheter deflection movement in a first direction with internal button 1512a, and an opposite direction with internal button 1512b), while actuator 1520 may be useful to control a second function (e.g., fine axial alignment and/or commissural alignment). Alternatively, the function of the actuators may be swapped and the orientation of the wheel may also, in addition or instead, be changed so that a wheel rotates forward and/or backward for fine axial translation in some examples. In some examples, multiple spring-return rocker switches may also be used (e.g., two spring-return rocker switches or three spring-return rocker switches).

In one variation to the above examples, FIG. 16 shows a delivery device handle 1600 having a housing 1605 and a first actuator 1610 in the form of a spring-return rocker switch having first internal button 1612a and second internal button 1612b, and additional actuators 1620a, 1620b in the form of push buttons disposed on either side of the first actuator 1610 for operating the delivery device. This embodiment is similar to a directional pad embodiment except that actuators 1620a, 1620b are isolated from first actuator 1610. In this example, first actuator 610 may include spring-return rocker so that pressing first or second internal buttons 1612a, 1612b toggle or activates a first primary function (e.g., catheter deflection), while actuators 1620a, 1620b may be useful to control another primary function (e.g., fine axial alignment and/or commissural alignment). Alternatively, the function of the actuators may be swapped and the orientation of the spring-return rocker may also, in addition or instead, be changed so that a spring-return rocker is oriented along the central axis of the handle.

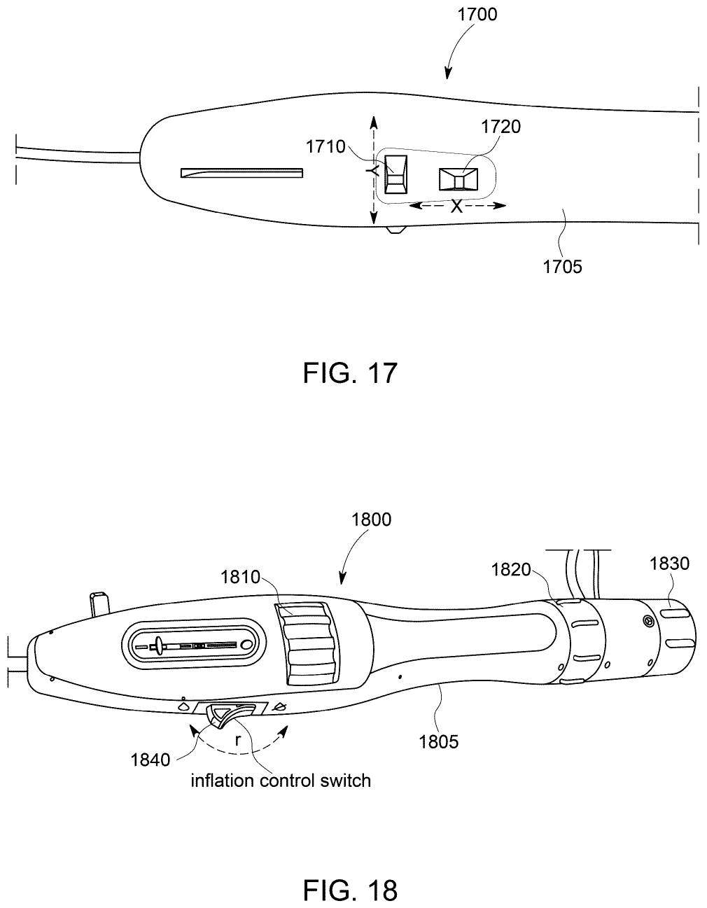

FIG. 17 illustrates an example of a delivery device handle 1700 having a housing 1705 and a first actuator 1710 in the form of a moveable toggle switch translatable up and/or down along a first axis y, and a second actuator 1720 in the form of a moveable toggle switch translatable forward and/or backward along a second axis x that is normal the first axis y. In this example, first actuator 1710 may control a first primary function (e.g., catheter deflection), while second actuator 1720 may be useful to control a second primary function (e.g., fine axial alignment and/or commissural alignment). Alternatively, the function of the actuators may be swapped and the orientations of the toggle switches may also, in addition or instead, be changed. In some examples, the two toggle switches are oriented in the same direction. In some examples, three toggle switches are provided, one for each of the three primary functions described above. Icons, indicators, markings, colors, and/or text may be used to signal to the user the primary function associated with each actuator.

FIG. 18 illustrates one example of a delivery device handle 1800 having a housing 1805 that is similar to delivery device handle of FIG. 12 above with some variations. In this example, handle 1800 includes a first actuator 1810 in the form of a first wheel disposed adjacent the distal end of the handle, a second actuator 1820 in the form of a second wheel disposed closer to the proximal end of the handle, and a third actuator 1830 in the form of a third wheel disposed proximal to the second actuator. Actuators 1810-1830 may function as previously described and may each operate one of the three primary functions as noted above. In addition, handle 1800 may also include a fourth actuator 1840 in the form of an inflation control switch. For example, for balloon-expandable TAVI, electronic inflation and/or deflation of the delivery balloon may be desirable and an inflation control switch may activate separate electromechanical syringe pumps for filling and/or evacuating the balloon during the delivery and/or implantation process. In some examples, fourth actuator 1840 may be in the form of a switch with a momentary slider function to ensure full user control (i.e., the actuator does nothing when released or when no external force is applied thereto). For example, the switch may be movable as shown with arrows “r” and pushing forward or distally on the switch may inflate the balloon, while pulling backward or proximally on the switch may deflate the balloon. In some examples, a neutral position is disposed between the two extreme positions, and the switch may be at rest in the neutral position absent any external force. In some examples, a momentary switch may facilitate a pause in inflation during the procedure based on procedural feedback, as desired by the operator.

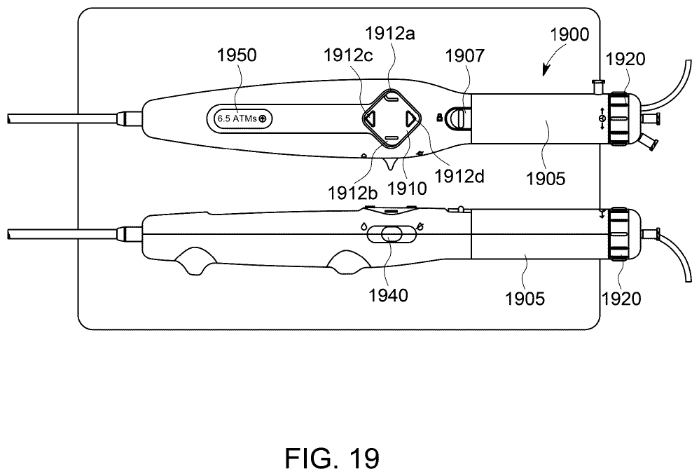

In one variation to the above examples, FIG. 19 shows a delivery device handle 1900 having a housing 1905 and a first actuator 1910 in the form of a directional pad. First actuator 1910 may include a first (“up”) internal button 1912a, an opposing second (“down”) internal button 1912b, a third (“left”) internal button 1912c and an opposing fourth (“right”) internal button 1912d, and each internal button may provide a discrete operation. It will be understood that the labels “up,” “down,” “left,” and “right” are only used to illustrate the positions of the internal button on the device in the orientation shown, and are not meant to limit the functionality of any button. In this example, first actuator 1910 may be used for two operations, such as deflection and fine adjustment control. For example, internal buttons 1912a, 1912b may deflect and straighten a catheter, respectively, and internal buttons 1912c, 1912d may provide fine control adjustment (e.g., forward and backward movement of the catheter). Delivery device handle 1900 may include a slideable lock button 1907 to limit movement of any one of the actuators (e.g., first actuator 1910). An additional actuator 1920 in the form of a wheel may also be included for operating the delivery device, and in some examples, actuator 1920 may be used to control commissural alignment. In addition, handle 1900 may also include an actuator 1940 in the form of a slideable inflation control switch. For example, for balloon-expandable TAVI, electronic inflation and/or deflation of the delivery balloon may be desirable and an inflation control switch may activate separate electromechanical syringe pumps for filling and/or evacuating the balloon during the delivery and/or implantation process. In some examples, actuator 1940 may be in the form of a switch with a momentary slider function to ensure full user control (i.e., the actuator does nothing when released or when no external force is applied thereto).

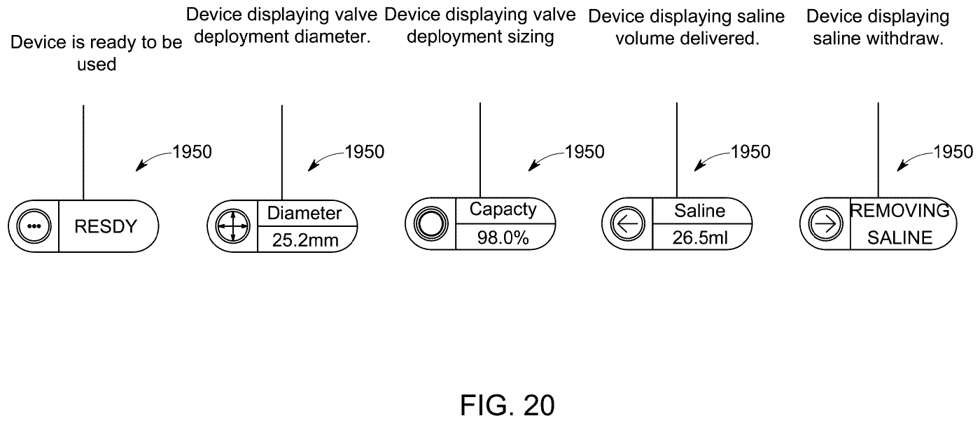

Optionally, delivery device handle 1900 may include a display window 1950. Display window 1950 may include an electronic display to convey pertinent information to the operator relating to balloon pressure, sizing, status, error codes or messages, stage of procedure or other similar information. Display window 1950 may include an LCD display or an E-ink display that is in communication with a pressure sensor or a semi-automatic inflation device or other data gathering component. FIG. 19 shows a display window 1950 that indicates a pressure with an arrow that indicates whether the pressure is increasing or decreasing. FIG. 20 shows several different types of information that can be presented in display window 1950. The examples shown in FIG. 20 are merely illustrative, and it will be understood that any combination of this information can be presented and that additional or different types of information may be presented. In some examples, display window 1950 may present a status of the device (e.g., “READY,” “INITIALIZING,” “ERROR,” “RESET REQUIRED,” etc.), a valve deployment diameter, a valve deployment sizing or capacity, such as that shown in percentage, the amount of saline and/or contrast delivered, and/or the status of the saline or inflation medium (e.g., “REMOVING SALINE,” “INTRODUCING SALINE,” etc.). The user interface may also include an icon that corresponds to the type of message being displayed. Several examples of icons are provided including status (shown as three dots), diameter (shown as a circle with four arrows), capacity (shown as concentric dashed and solid circles), saline volume delivered (shown with a left arrow indicating movement into the balloon), or saline withdrawal (shown with a right arrow indicating movement out of the balloon toward the handle).

Certain optional features may also be added to any of the embodiments described herein. For example, additional handle features to enable vibration, buzzing, sounds, and/or other tactile feedback to communicate the status of device actuation, procedural feedback, and/or other relevant information regarding patient wellness, any of the primary functions described above or some other parameter relating to the procedure timing, effectiveness and/or status. Inputs for these tactile modes of feedback may be from the delivery device itself, an accessory device, or other existing infrastructure in the operating room and/or a catheterization laboratory. Inputs may also come from wired and/or wireless communication sources that are in communication with the implant and/or the delivery device.

In some examples, for balloon expanding transcatheter aortic valve implantation, a sensor for measuring pressure inside the balloon may be mounted proximal to the balloon component itself, for example inside the delivery system control handle. This pressure sensor may enable closed loop feedback for potential changes to the inflation and/or deflation action based on pressure value or pressure changes, such as proximity to rated burst pressure (for safety purposes), and/or inflation or deflation rates and/or times (for potential optimization of procedural outcomes). These downstream pressures could also be offset from actual balloon pressures based on a known relationship or system algorithm. In some examples, the pressure readings or values may also be measured and stored on the delivery device or transmitted to a remote server or memory and used to further optimize procedures, retrieve data relating to a particular procedure or better understand variability between patients.

It will be understood that any of the control features presented above may be linked to a DC motor, stepper motor, servo motor, solenoid, and/or other electromagnetic machine to enable action. Additionally, any of the control features presented above may be actuated via toggle switch, slide switch, rocker switch, push button switch, potentiometer, tactile switch, capacitive switch, operator voice or speech, and/or other input control to enable user input to power the motor control. The controls could be maintained or momentary in nature. Additionally, any of these control features may utilize wired or wireless communication. Moreover, any of these control features may include the ability to change speed of actuation via potentiometer, additional detents, or additional button (e.g., tempo or turbo mode) to enable more user control based on procedural feedback.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A delivery device comprising:
a delivery catheter; and
a handle coupled to the delivery catheter, the handle having a housing and including at least two actuators, each of the at least two actuators being configured to control one of three primary functions, the three primary functions including (1) delivery catheter deflection, (2) delivery catheter axial position fine adjustment, and (3) delivery catheter rotation to achieve native valve commissural alignment.

2. The delivery device of claim 1, wherein the at least two actuators are integrated into a directional pad having a first set of two internal buttons aligned along a first axis, and a second set of two internal buttons aligned along a second axis normal to the first axis.

3. The delivery device of claim 2, wherein the first set of two internal buttons controls a first of the three primary functions, and the second set of internal buttons controls another of the three primary functions.

4. The delivery device of claim 1, wherein the at least two actuators include a wheel and two push buttons, the two push buttons including one push button disposed on either side of the wheel.

5. The delivery device of claim 1, wherein the at least two actuators include a spring-return rocker switch having two internal buttons, and a wheel spaced from the spring-return rocker switch.

6. The delivery device of claim 1, wherein the at least two actuators include a spring-return rocker switch having two internal buttons, and two additional independent push buttons, each of the two independent push buttons being disposed on a side of the spring-return rocker switch.

7. The delivery device of claim 1, wherein the at least two actuators include a first movable toggle switch and a second movable toggle switch.

8. The delivery device of claim 7, wherein the first movable toggle switch is configured to translate along a first axis, and the second movable toggle switch is configured to translate along a second axis normal to the first axis.

9. The delivery device of claim 1, wherein the at least two actuators include three separate wheels, each of the three separate wheels being dedicated to one of three primary functions.

10. The delivery device of claim 9, further comprising a momentary inflation control switch movable in a first direction to inflate a delivery balloon and movable in a second direction to deflate the delivery balloon.

11. A delivery device comprising:
a delivery catheter; and
a handle having a housing and including:
a first actuator configured to control delivery catheter rotation relative to the handle to achieve native valve commissural alignment; and
a second actuator configured to control (a) delivery catheter deflection, or (b) delivery catheter axial position fine adjustment.

12. The delivery device of claim 11, wherein the second actuator is configured to control delivery catheter deflection.

13. The delivery device of claim 11, wherein the second actuator is configured to control delivery catheter axial position fine adjustment.

14. The delivery device of claim 11, wherein the first actuator and the second actuators are integrated into a directional pad having a first set of two internal buttons aligned along a first axis, and a second set of two internal buttons aligned along a second axis normal to the first axis.

15. The delivery device of claim 11, wherein the second actuator includes a wheel and the first actuator includes two separate push buttons, the two separate push buttons including one push button disposed on either side of the wheel.

16. The delivery device of claim 11, wherein the second actuator comprises a spring-return rocker switch having two internal buttons, and the first actuator comprises a wheel spaced from the spring-return rocker switch.

17. The delivery device of claim 11, wherein the second actuator comprises a spring-return rocker switch having two internal buttons, and the first actuator comprise two additional independent push buttons, the two independent push buttons being disposed on either side of the spring-return rocker switch.

18. The delivery device of claim 11, wherein the first actuator comprises a first movable toggle switch and the second actuator comprises a second movable toggle switch.

19. The delivery device of claim 18, wherein the first movable toggle switch is configured to translate along a first axis, and the second moveable toggle switch is configured to translate along a second axis normal to the first axis.

20. The delivery device of claim 11, further comprising a display window disposed on a same surface as the first actuator, the display window being capable of providing information relating to at least one of valve deployment diameter, valve deployment sizing, and volume of inflation media delivered.

21. The delivery device of claim 11, wherein the handle further includes an alerting feature configured to vibrate, buzz, make a sound, and/or provide tactile feedback to communicate a status of device actuation, procedural feedback, and/or other relevant information regarding patient wellness.

22. The delivery device of claim 11, further comprising a toggle configured to change a speed of actuation.
